(19) United States
(12) Patent Application Publication
(10) Pub. No.: US 2024/0090967 A1
(43) Pub. Date: Mar. 21, 2024

(54) METHOD AND APPARATUS FOR SURGICAL DRILL STOP PLACEMENT

(71) Applicant: INNOVATE OUR WORLD CONSULTANTS, LLC, Houston, TX (US)

(72) Inventor: Ray King, Houston, TX (US)

(21) Appl. No.: 18/264,407

(22) PCT Filed: Feb. 5, 2022
(86) PCT No.: PCT/US2022/015386

§ 371 Date: Aug. 4, 2023

(51) Int. Cl. A61B 90/00; A61B 10/02

(52) U.S. Cl. CPC A61B 90/03; A61B 10/025; A61B 2090/034; A61B 2090/062

(57) ABSTRACT

The present invention relates generally to methods and systems for an adjustable depth stop, which has additional optional features of tissue retraction, visualization via transparent or semi-transparent material and/or window(s) on its size, precise gradation of depth by rotation indicated on side of the device, windows on the side for irrigation while traversing, and ability to utilize this device in settings of biopsy, coagulation, laser ablation, and other non-drill-related uses whereupon depth needs to be achieved, and going past-point would be problematic, in a device (such as an catheter, electrode, or needle, but not intended for a drill) which cannot be easily “overpowered” by typically applied forces (e.g. downward pressure by typical user). This depth stop could have a nested, telescoping feature as well, so that it may be increased in length to many-fold its original length. It may be utilized as a device to be manually set, or automatically set (e.g. utilizing a robotic feature).

### CROSS-REFERENCE TO RELATED APPLICATIONS

This Patent Cooperation Treaty international application claims the benefit and priority of U.S. provisional patent application Ser. No. 63/146,065, filed Feb. 5, 2021, entitled “METHOD AND APPARATUS FOR SURGICAL DRILL STOP PLACEMENT”, the contents of which is hereby incorporated by reference in its entirety.

### FIELD OF THE INVENTION

This invention relates generally to a depth stop which prevents a surgically placed device (e.g. electrode, catheter, needle, other than a drill) from penetrating too far or plunging beyond a desirable depth, such as into a tissue (e.g. into the bone or beyond the bone), an organ (e.g. the brain) or a blood vessel/nerve/organ/other tissue, and further relates to methods and systems for using such a depth stop.

### BACKGROUND OF THE INVENTION

Devices such as electrodes, catheters, or needles are placed to a certain depth, but when that desired depth has been reached, they typically employ a stopper to prevent excessive travel beyond the intended length or depth. To facilitate this, current devices (e.g. for neurosurgical or orthopedic procedures) have a “stopper.” The most common version, for neurosurgical procedures, are found in the “Stereotactic biopsy needle trays” or other stereotactic trays. The biopsy needle is typically accompanied by adjustable depth stops which comprise a plastic ring with a hole drilled on its side, through which a thumbscrew (or in some cases a set screw) is placed (and tightens or loosens manually using fingers, or a tool such as via an Allen key). This depth stop has a goal of preventing undesirable plunging past the intended target, so can be so adjusted by the user with the goal to accomplish this.

In a percentage of cases (ranging up to 10%, however, objects may be placed at depths “past point” exceeding the intended depth, and hence a depth stop that cannot be overpowered (e.g. the United States Food and Drug Administration (FDA) standard of predicate devices of 0.5 pounds for grasping a depth electrode) easily is desirable, to further minimize or completely negate plunging risk.

### SUMMARY OF THE INVENTION

This invention relates generally to a depth stop which prevents a surgical device (catheter, electrode, needle), from penetrating too far or plunging beyond a desirable depth, such as into a tissue (e.g. into the bone or beyond the bone), an organ (e.g. the brain) or a blood vessel/nerve/organ/other tissue, and further relates to methods and systems for using such a depth stop. In general, a depth stop may include a cylindrical member through which a device (catheter, electrode, needle, other than a drill) may pass through. The cylindrical member may generally be wider in diameter than the depth stop to a degree that the cylindrical member may abut against the surface being penetrated to stop penetration of the device during use after a given distance of travel. The depth stop may generally be utilized by attachment to the device or device placer's body, attachment to an existing primary depth stop, or held against manually or attached to a body surface during placement. The depth stop may be constructed of any appropriate material, such as plastic, metal, composite, and/or a combination thereof.

In one exemplary aspect, the depth stop may include an adjustability feature to change the amount of depth before the stop abuts a surface to control the depth of penetration of the device. In some embodiments, the adjustability feature includes a depth adjusting insert that may interface with the cylindrical member of the depth stop and act to adjust the overall length of the depth stop to affect the amount of depth of penetration when placing such a device. The adjusting insert may, for example, thread into corresponding threads in the cylindrical member and the overall length of the depth stop may then be adjusted by rotating the adjusting insert in the appropriate direction to thread or unthread it from the cylindrical member. In general, the threading may produce enough friction and/or other resistance such that the adjusting insert remains at the given degree of insertion into the cylindrical member during use. The depth stop may thus remain adjustable, but may be tightened to its desired position without relying upon other forms of locking, such as a side-bracing set screw. This may be desirable such that greater than 0.5 pounds of pressure (e.g. the metric used by companies with electrode grasping sufficiency) is not adequate to displace it—ideally, a depth stop capable of withstanding about 10 to 100 times this weight or more, making it impractical for a device to plunge may be desirable. In some embodiments, an additional locking mechanism, such as a locking collar, a set screw or any other appropriate locking mechanism may also be included for added assurance in maintaining the desired depth setting.

In some embodiments, the threading may be designed such that the depth setting is metered based on the number of full rotations of the adjusting insert. For example, a full rotation may increase or lower, depending on the direction, the overall length of the depth stop by a set amount, such as 1 mm or any other desirable amount of depth.

In some embodiments, the depth stop may include markings or other forms of graduations such that a user may visually assess the depth setting.

In another aspect of the invention, the depth stop may include a visualization and/or access feature(s). In general, a visualization feature may provide the user with a view of the interior of the depth stop, such as, for example, to see the immediate area being traversed. In some embodiments, a window or other aperture may be utilized as a visualization feature. A window or aperture may also provide access to the area, such as for irrigation during device passage. The window or aperture may also be generally placed proximal to the end of the depth stop that abuts the surface during device placement. In some embodiments, the window or aperture may be positioned on the depth stop such that it is elevated off the surface during use, for example, to prevent tissue from blocking or entering the window or aperture, such as the scalp or skin.

In some embodiments, at least a portion of the depth stop may be constructed from a transparent or translucent material such that a user may visualize through the material of the depth stop.

In another aspect of the invention, the depth stop may include features for providing tissue retraction during use. For example, it may be desirable to prevent tissue from entering the device passage area, such as to prevent tissue from contacting the device being placed and being damaged/chafed. In some embodiments, the depth stop may include a lip or other protruding formation that may, for example, be utilized to push away tissue and/or be inserted under the tissue to retract it away from the placement area, such as, for example, inserting under skin/scalp.

In another aspect of the invention, the depth stop may include features for aiding in the user holding and/or stabilizing the depth stop during use. In some embodiments, the depth stop may include finger impressions or other gripping features such that the user may hold it as needed, for stabilization; alternatively, it may be screwed onto the primary depth stop or the placement device's housing, or used free-hand without stabilization (e.g. with manual or power placers of devices).

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE DRAWINGS

FIG. 1 illustrates a depth stop of the present invention, as applied, for example, to a bone biopsy needle, in closed form;

FIG. 1a illustrates the cylindrical member of the depth stop as applied, for example, to the bone biopsy needle;

FIG. 1b illustrates a depth stop of the present invention, as applied, for example, to a bone biopsy needle, in closed form;

FIG. 1c illustrates the cylindrical member of the depth stop of the present invention, as applied, for example, to a bone biopsy needle;

FIGS. 2 and 2a illustrates a depth stop with a nested telescoping feature, whereby increase nearly three-fold or more may be allowed; and

FIG. 3 illustrates a prior art surgical device (biopsy needle) with a primary depth stop.

### DETAILED DESCRIPTION OF THE INVENTION

The detailed description set forth below is intended as a description of the presently exemplified systems, devices and methods provided in accordance with aspects of the present invention and are not intended to represent the only forms in which the present invention may be prepared or utilized. It is to be understood, rather, that the same or equivalent functions and components may be accomplished by different embodiments that are also intended to be encompassed within the spirit and scope of the invention. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplary methods, devices and materials are now described. All publications mentioned herein are incorporated herein by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

This invention relates generally to a depth stop which prevents a device (e.g. catheter, electrode, needle, but not a drill) from penetrating too far or plunging beyond a desirable depth, such as into a tissue (e.g. into the bone or beyond the bone), an organ (e.g. the brain) or a blood vessel/nerve/organ/other tissue, and further relates to methods and systems for using such a depth stop.

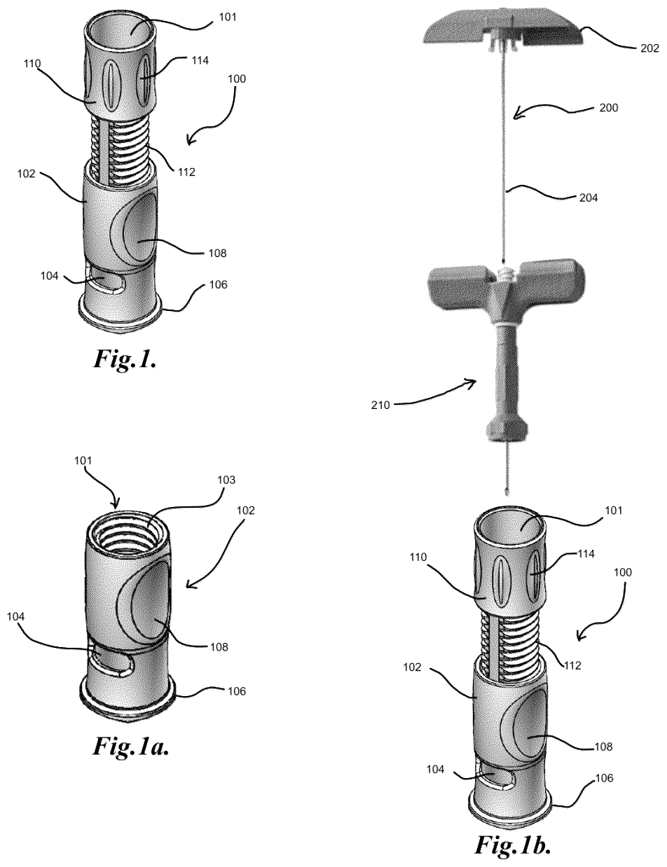

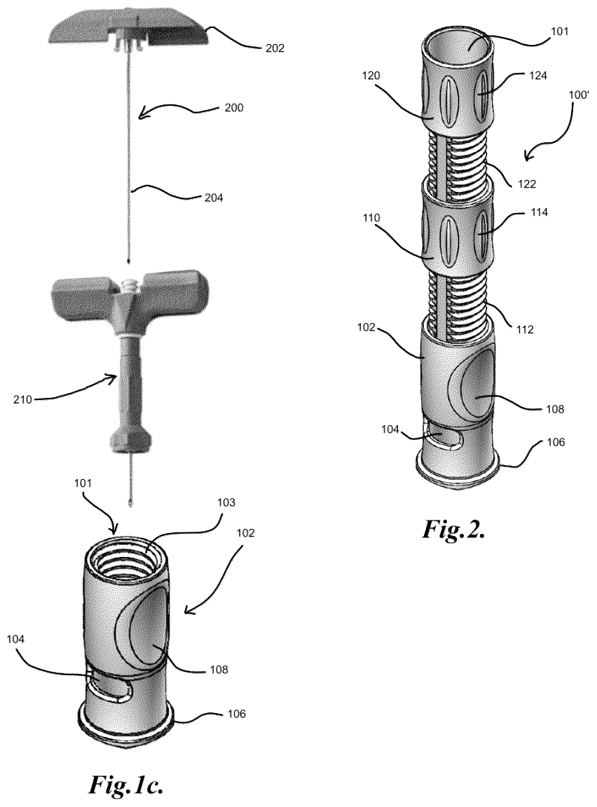

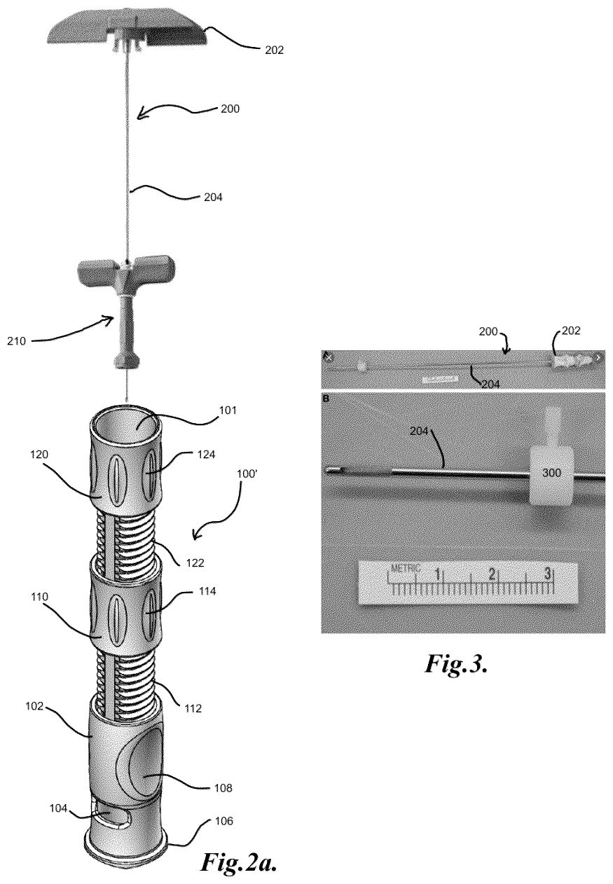

In general, as illustrated in FIG. 1, a depth stop 100 may include a cylindrical member 102, shown more specifically in FIG. 1a, through which a device may pass through via a channel 101. The cylindrical member 102 may generally be wider in diameter than the device to a degree that the cylindrical member 100 may abut against the surface being traversed to stop penetration of the device during use after a given distance of travel. FIG. 1B illustrates a placed device 200 with a device body 202 and tip 204 shown as significantly narrower than the cylindrical member 102. The depth stop 100 may generally be utilized by attachment to the device body 202, attachment to an existing primary portion of a medical device, such as a portion of a device (as illustrated with portion 210 in FIGS. 1b, 1c and 2a), such as the primary depth stop 300 illustrated in FIG. 3, or held against manually or attached to a body surface during placement.

The depth stop 100 may be constructed of any appropriate material, such as plastic, metal, composite, and/or a combination thereof. For example, in general, the various components of the depth stop 100 may be made from appropriate materials, such as polymers, metals, composites, and/or any other appropriate material or combinations thereof. Suitable polymers may include, but are not limited to, polyethylene; polypropylene; polybutylene; polystyrene; polyester; polytetrafluoroethylene (PTFE); acrylic polymers; polyvinylchloride; Acetal polymers such as polyoxymethylene or Delrin (available from DuPont Company); natural or synthetic rubber; polyamide, or other high temperature polymers such as polyetherimide like ULTEM®, a polymeric alloy such as Xenoy® resin, which is a composite of polycarbonate and polybutyleneterephthalate, Lexan® plastic, which is a copolymer of polycarbonate and isophthalate terephthalate resorcinol resin (all available from GE Plastics); liquid crystal polymers, such as an aromatic polyester or an aromatic polyester amide containing, as a constituent, at least one compound selected from the group consisting of an aromatic hydroxycarboxylic acid (such as hydroxybenzoate (rigid monomer), hydroxynaphthoate (flexible monomer), an aromatic hydroxyamine and an aromatic diamine, (exemplified in U.S. Pat. Nos. 6,242,063, 6,274,242, 6,643,552 and 6,797,198, the contents of which are incorporated herein by reference), polyesterimide anhydrides with terminal anhydride group or lateral anhydrides (exemplified in U.S. Pat. No. 6,730,377, the content of which is incorporated herein by reference) or combinations thereof. Some of these materials are recyclable or may be made to be recyclable. Compostable or biodegradable materials may also be used and may include any biodegradable or biocompostable polyesters such as a polylactic acid resin (comprising L-lactic acid and D-lactic acid) and polyglycolic acid (PGA), polyhydroxyvalerate/hydroxybutyrate resin (PHBV) (copolymer of 3-hydroxy butyric acid and 3-hydroxy pentanoic acid (3-hydroxy valeric acid) and polyhydroxyalkanoate (PHA) copolymers, and polyester/urethane resin. Some non-compostable or non-biodegradable materials may also be made compostable or biodegradable by the addition of certain additives, for example, any oxo-biodegradable additive such as D2W™ supplied by (Symphony Environmental, Borehamwood, United Kingdom) and TDPA® manufactured by EPI Environmental Products Inc. Vancouver, British Columbia, Canada. Compostable or biodegradable materials may also be desirable for disposable versions or portions of the depth stop 100. In addition, any polymeric composite such as engineering prepregs or composites, which are polymers filled with pigments, carbon particles, silica, glass fibers, or mixtures thereof may also be used. For example, a blend of polycarbonate and ABS (Acrylonitrile Butadiene Styrene) may be used for the housing. For further example, carbon-fiber and/or glass-fiber reinforced plastic may also be used. Useful metals or metallic materials may include metal and metal alloys such as aluminum, steel, stainless steel, nickel titanium alloys, shape memory alloys and so on. In general, for certain applications which involve imaging, the depth stop 100 may be made from magnetic resonance imaging (MRI) compatible or X-ray/computerized tomography (CT) compatible materials, such as polymers, titanium, aluminum, etc.

In one exemplary aspect, the depth stop 100 may include an adjustability feature to change the amount of depth before the stop abuts a surface to control the depth of penetration of the device 204. In some embodiments, the adjustability feature includes a depth adjusting insert that may interface with the cylindrical member of the depth stop and act to adjust the overall length of the depth stop to affect the amount of depth of penetration when placing a device, as illustrated with the adjusting insert 110 in FIGS. 1 and 1a. The adjusting insert 110 may, for example, thread into corresponding threads in the cylindrical member 102 and the overall length of the depth stop 100 may then be adjusted by rotating the adjusting insert 110 in the appropriate direction to thread or unthread it from the cylindrical member 102. FIGS. 1 and 1a illustrate a threading adjusting insert 110 that threads into cylindrical member 102 via threads 112 and 103, respectively. In general, the threading, such as between the threads 103, 112, may produce enough friction and/or other resistance such that the adjusting insert 110 remains at the given degree of insertion into the cylindrical member 102 during use. The depth stop 100 may thus remain adjustable, but may be tightened to its desired position without relying upon other forms of locking, such as a side-bracing set screw. This may be desirable such that greater than five pounds of pressure (e.g. the FDA standard) is not adequate to displace it—ideally, a depth stop 100 capable of withstanding five to ten times (or more-fold) this weight or more, making it impractical for a non-drill device to plunge may be desirable. In some embodiments, an additional locking mechanism, such as a locking collar, a set screw or any other appropriate locking mechanism may also be included for added assurance in maintaining the desired depth setting.

In some embodiments, the threading, such as between threads 103, 112, may be designed such that the depth setting is metered based on the number of full rotations of the adjusting insert 110. For example, a full rotation may increase or lower, depending on the direction, the overall length of the depth stop 100 by a set amount, such as 1 mm or any other desirable amount of depth.

In some embodiments, the depth stop 100 may include markings or other forms of graduations such that a user may visually assess the depth setting.

In some embodiments, the depth stop may include multiple adjusting inserts, as illustrated with depth stop 100′ in FIGS. 2 and 2a with adjusting inserts 110 and 120. This may generally increase the range of depth settings. The adjusting inserts may be designed to be modular, such that additional adjusting inserts may be added on as needed to increase the total depth setting available for use.

In other embodiments, the depth stop 100 may be utilized without the adjusting insert 110 if the length of the cylindrical member 102 is appropriate alone, as illustrated in FIG. 1c.

In another aspect of the invention, the depth stop 100 may include a visualization and/or access feature(s). In general, a visualization feature may provide the user with a view of the interior of the depth stop, such as, for example, to see the immediate area being traversed. In some embodiments, a window or other aperture may be utilized as a visualization feature. A window or aperture may also provide access to the area, such as for irrigation for cleaning or cooling, determination of bleeding, or removing biopsy sample proximally without removing the entire needle. The window or aperture may also be generally placed proximal to the end of the depth stop that abuts the surface during placement. In some embodiments, the window or aperture may be positioned on the depth stop such that it is elevated off the surface during use, for example, to prevent tissue from blocking or entering the window or aperture, such as the scalp or skin. FIGS. 1, 1a, 1b, 1c, 2 and 2a illustrate a window 104 for visualization/access.

In some embodiments, at least a portion of the depth stop 100 may be constructed from a transparent or translucent material such that a user may visualize through the material of the depth stop 100.

In another aspect of the invention, the depth stop 100 may include features for providing tissue retraction during use. For example, it may be desirable to prevent tissue from entering the placement area, such as to prevent tissue from contacting the device and being damaged/chafed. In some embodiments, the depth stop 100 may include a lip or other protruding formation, such as the lip 106 illustrated in FIGS. 1, 1a, 1b, 1c, 2 and 2a, that may, for example, be utilized to push away tissue and/or be inserted under the tissue to retract it away from the traversing area, such as, for example, inserting under skin/scalp.

In another aspect of the invention, the depth stop 100 may include features for aiding in the user holding and/or stabilizing the depth stop 100 during use. In some embodiments, the depth stop 100 may include finger impressions or other gripping features, as illustrated with gripping features 108 and 114 in FIGS. 1 and 1a, such that the user may hold it as needed, for stabilization; alternatively, it may be screwed onto the primary depth stop 300 or the device housing 202, or used free-hand without stabilization (e.g. on manually controlled or automatically controlled devices, including robotic devices).

### Examples: Use of Depth Stop

Stereotactic brain biopsy involves making a hole in the skull, taking care to avoid not traversing too deep (as the dura or brain may be penetrated past the desired target, resulting in permanent injury)—with present traversing being somewhat controlled by a “depth stop” which is a plastic or nylon cannulated cylinder and a plastic set screw on its side tightened to abut the metal device, this is highly user dependent (in terms of how tightly or loosely the set screw was originally tightened, and how much downward pressure was placed on the device when traversing)—with 0.5 pounds being the threshold of predicate devices (such as holding in a deep brain stimulation catheter, per Medtronic). Unfortunately, plunging occurs (reflected by personal experiences and the published peer-reviewed literature) for this scenario. In bone biopsy cases including extremity or spine cases, similar protection against traversing too far may be required, to avoid injury to distal blood vessels, nerves, or other tissue. Creating the entire structure with gradations to measure the depth, as well as of a clear material vs. opaque, would allow visualization inside; in addition, a longer version of this could be used for spine procedures whereby the pedicle cannulating device may be placed with length of device placed through it (e.g. biopsy needle) limited to not exit the vertebral body anteriorly (similarly for kyphoplasty).

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, or feature, while functionally limited to traversing). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention, while maintaining its use to the indication of traversing.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A depth stop for a medical device comprising:
a cylindrical member having a channel adapted to receive a penetrating medical device therethrough; and
an adjusting insert adapted to interface with said cylindrical member to alter the length of said depth stop;
wherein said depth stop is adapted to withstand an applied force of at least five pounds of pressure.

2. The depth stop of claim 1, wherein said adjusting insert interfaces with said cylindrical member via threadings.

3. The depth stop of claim 1, further comprising a visualization aperture.

4. The depth stop of claim 1, further comprising a tissue retraction feature.

5. The depth stop of claim 1, further comprising at least one additional adjusting insert for increasing a range of depth settings of said depth stop.

6. The depth stop of claim 1, further comprising a manual, automatic or robotic-interfacing adjustment setting.

7. A method for using a depth stop with a medical device comprising:
providing a depth stop comprising:
a cylindrical member having a channel adapted to receive a medical device therethrough; and
an adjusting insert adapted to interface with said cylindrical member to alter the length of said depth stop;
adjusting said depth stop via said adjusting insert to set a desired insertion depth;
inserting a medical device through said channel to insert into a penetration site until a portion of said medical device abuts against said depth stop and said depth stop abuts against a surface of said body tissue surrounding said penetration site to stop the insertion of said medical device;
wherein said depth stop is adapted to withstand an applied force of at least five pounds of pressure and said medical device comprises a non-drill device.
